(19) United States
(12) Patent Application Publication
(10) Pub. No.: US 2009/0318670 A1
(43) Pub. Date: Dec. 24, 2009

(54) Separation of Proteins from Grasses Integrated with Ammonia Fiber Explosion (AFEX) Pretreatment and Cellulose Hydrolysis

(76) Inventors: Bruce E. Dale, Mason, MI (US); Bryan Bals, East Lansing, MI (US); Venkatesh Balan, East Lansing, MI (US)

Correspondence Address: IAN C. McLEOD, P.C., 2190 COMMONS PARKWAY, OKEMOS, MI, 48864, US

(21) Appl. No.: 12/226,850

(22) PCT Filed: Apr. 30, 2007
(86) PCT No.: PCT/US07/10410

§ 371 Date: Jun. 22, 2009

Related U.S. Application Data
(60) Provisional application No. 60/796,401, filed on May 1, 2006.

(51) Int. Cl. C07K 1/00

(52) U.S. Cl. 530/344

(57) ABSTRACT

A process for extracting an aqueous ammonium hydroxide solution from a plant biomass after an Ammonia Fiber Explosion (AFEX) process step, is described. The proteins can be separated before or after a hydrolysis of sugar precursors (carbohydrates) from the biomass to produce sugars for fermentation to produce ethanol. The proteins are useful as animal feeds because of their amino acid food values.

### CROSS-REFERENCE TO RELATED APPLICATIONS

This application claims benefit to U.S. Provisional Application Ser. No. 60/796,401, filed May 1, 2006, which is incorporated herein by reference in its entirety.

### STATEMENT REGARDING FEDERALLY SPONSORED RESEARCH OR DEVELOPMENT

This research was under a grant from the United States Department of Energy (USDOE) Contract No. DE-FG36-04GO-14220. THE U.S. GOVERNMENT HAS CERTAIN RIGHTS TO THIS INVENTION.

### BACKGROUND OF THE INVENTION

(1) Field of the Invention

The present invention relates to a process for extracting proteins from an Ammonia Fiber Explosion (AFEX) process pretreated plant biomass. The process uses a relatively dilute ammonium hydroxide solution to extract the proteins. The process is part of a process for hydrolyzing extracted sugar precursors (carbohydrates) from plants into sugars which are used for fermentation to produce ethanol.

(2) Description of the Related Art

Recent concerns about the environmental, political, and economic impact of oil use have spurred renewed interest in alternative fuels for transportation. Ethanol derived from cellulosic feedstocks such as agricultural waste, wood chips, municipal waste, or forages is one particularly attractive alternative because it is domestically available, renewable, and can potentially reduce greenhouse gas emissions (1). Although early biorefineries will likely use agricultural residue as feedstocks due to their low cost, dedicated energy crops will be necessary to reach the very high levels of ethanol production proposed in various studies (2).

Switchgrass (Panicum vergatum) is a model herbaceous energy crop, and is attractive as a feed stock due to several favorable characteristics: high crop yields, low soil erosion, low water, fertilizer and pesticide requirements, ability to sequester carbon, and high genetic variability (2-3). Ample research has been conducted from the agricultural perspective, providing a foundation for further investigation and optimization using switchgrass for ethanol production (3).

In order to ferment the carbohydrates in cellulosic feedstocks into ethanol, they must first be broken down into their component sugars. However, yields from enzymatic hydrolysis are low unless the biomass first undergoes a pretreatment process. The best method to improve the efficiency of the hydrolysis is the ammonia fiber expansion (AFEX) process. Concentrated ammonia is added to the biomass under high pressure and moderate temperatures, held for a residence time for preferably about 5 minutes, before rapidly releasing the pressure. This process decrystallizes the cellulose, hydrolyzes hemicellulose, removes and depolymerizes lignin, and increases the size of micropores on the cellulose surface, thereby significantly increasing the rate of enzymatic hydrolysis (4). Previous work has shown this process to give near theoretical yields of glucose on different types of agricultural residue (5-6) and grasses (7-8). In particular, previous work has shown conversions of over 90% of the glucan and 70% of the xylan for switchgrass (9).

The prior art in the pretreatment of plant biomass with anhydrous liquid ammonia or ammonium hydroxide solutions in an AFEX process is extensive. Illustrative are the following patents and literature references which are incorporated herein by reference in their entireties.

U.S. Pat. No. 4,600,590 to Dale.
U.S. Pat. No. 4,644,060 to Chou.
U.S. Pat. No. 5,037,663 to Dale.
U.S. Pat. No. 5,171,592 to Holtzapple et al.
U.S. Pat. No. 5,865,898 to Holtzapple et al.
U.S. Pat. No. 5,939,544 to Karstens et al.
U.S. Pat. No. 5,473,061 to Bredereck et al.
U.S. Pat. No. 6,416,621 to Karstens.
U.S. Pat. No. 6,106,888 to Dale et al.
U.S. Pat. No. 6,176,176 to Dale et al.
Felix, A., et al., Anim. Prod. 51, 47-61 (1990).

### Waiss, A. C., Jr., et al., Journal of Animal Science, 35 No. 1, 109-112 (1972).

Although the structural carbohydrates in lignocellulosic feedstocks is the largest component in plant biomass, several other components are present as well. In an ideal biorefinery, each component would be processed into value added products (10). In particular, proteins are a potentially valuable co-product which can be separated from the rest of the biomass and sold as animal feed or other value added products. Such a process could have numerous benefits, including potentially decreasing the cost of producing ethanol. Greene et al. estimate that extracting proteins from switchgrass in a mature biorefinery could reduce the selling price of ethanol by nearly 20% (2). Furthermore, an acre of switchgrass can produce at least as much protein as an acre of soybean, providing the opportunity to replace soy acreage with switchgrass, and thereby increasing the total amount of biofuels able to be produced in the United States without reducing the capacity to produce animal feed (2).

There have been no reported studies of extracting proteins from switchgrass, although several other types of biomass have been considered for production of protein concentrates (10-16). Dilute solutions of a strong alkali such as sodium hydroxide are generally used, with the pH between 8 and 12. Extractions generally range from 30 to 60 minutes at 10:1 or higher liquid to solid ratio. Protein yields varied considerably depending upon the types of biomass, generally resulting in high yields of protein from grains and moderate to low yields from leaf proteins. Studies with Atriplex leaves obtained only 41% of the total protein, while a pilot plant extracting proteins from alfalfa obtained 47% of the total protein (15-16). In general, it appears that simple extractions are not sufficient to obtain complete protein recovery from leafy biomass.

However, to date, very little research has been done into integrating a protein extraction process with ethanol production. De la Rosa (17) and Urribarri (18) found increases in protein yields from coastal bermudagrass and dwarf elephant grass, respectively, when undergoing ammonia pretreatment prior to extraction.

In the case of protein extraction, the dominant “old” approach has been to mechanically grind and squeeze freshly harvested leafy green plant material such as alfalfa (containing about 80% water) to produce a protein rich juice. This protein rich juice is then heated or otherwise processed to coagulate and precipitate proteins. These protein precipitates are further processed to produce protein feeds for animals and also human feeds. A residual “brown juice” is left behind following protein precipitation and contains a variety of solubles. Few efforts have been made in previous approaches to utilize this “brown juice” or similar products. Nor have previous approaches attempted to increase the value of the residual fiber rich stream from which these proteins were derived.

### OBJECTS

It is therefore an object of the present invention to provide a process which enables the efficient extraction of proteins from a plant biomass along with sugar precursors (carbohydrates) used for production of ethanol. These and other objects will become increasingly apparent by reference to the following description and the drawings.

### SUMMARY OF THE INVENTION

The present invention relates to a process for extracting plant proteins from a lignocellulosic plant biomass comprising: (a) providing a harvested lignocellulosic plant biomass; (b) treating the plant biomass with an Ammonia Fiber Explosion (AFEX) process to provide a treated plant biomass; and (c) extracting proteins in the treated plant biomass with an aqueous alkaline ammonium hydroxide solution comprising up to about 3% by weight NH₄OH to provide the extracted proteins in the solution. Preferably, the plant is a monocot. More preferably, the monocot is rice or maize. Further, preferably the plant material is switchgrass. Still further, the pH is preferably above about 8. Further still, the proteins are separated from the solution by precipitation or ultrafiltration. Further, the extracting is after a hydrolysis step in the plant biomass, after step (b), to produce sugars from sugar precursors in the biomass. Still further, the extracting of the proteins is before a hydrolysis step in the plant biomass, after step (b), to produce sugars from sugar precursors in the biomass and optionally in addition after the hydrolysis step.

The present invention relates to a process for isolating plant proteins from a lignocellulosic plant biomass comprising: (a) providing a harvested lignocellulosic plant biomass; (b) treating the plant biomass with an Ammonia Fiber Explosion (AFEX) process to provide a treated plant biomass; (c) soaking the treated plant biomass in an alkaline aqueous solution of ammonium hydroxide at 25° to 70° C. to provide a soaked plant biomass in the solution; (d) extracting the solution from the soaked plant biomass in step (c); and (e) separating crude proteins from the solution of step (d) so as to provide isolated plant proteins from the plant biomass. Preferably the plant is a monocot. Most preferably the monocot is switchgrass, rice or maize. Further, the plant biomass is preferably switchgrass. Further, preferably the pH is above about 8. Still further, the proteins are separated from the solution by precipitation or ultrafiltration. Further still, the proteins are separated after a hydrolysis step in the plant biomass, after step (b), to produce sugars from structural carbohydrates in the biomass. Finally, preferably the proteins are separated before a hydrolysis step in the biomass, after step (b), to produce sugars from structural carbohydrates and optionally in addition after the hydrolysis step.

Proteins from lignocellulosic biomass such as grasses can provide an economic benefit to biorefineries by providing a valuable co-product to ethanol processing. This invention provides a process for extracting these proteins in line before the ethanol production, and after an Ammonia Fiber Explosion (AFEX) pretreatment to remove the protein. The grasses are extracted with an aqueous ammonium hydroxide solution. The extract can undergo enzymatic hydrolysis to convert its cellulose and hemicellulose to simple sugars before or after the removal of the proteins. After hydrolysis, the proteins released during this step are separated from the sugars by ultrafiltration or precipitation. The remaining solid residue undergoes a simulated crossflow extraction using an aqueous ammonia solution as the solvent, where the remaining protein is recovered. This process can remove up to 99% of the protein from the biomass, indicating a high yield is attainable. The ammonia used can be recycled into the AFEX process. The protein extract is sold as animal feed or recycled back into hydrolysis.

### BRIEF DESCRIPTION OF THE DRAWINGS

FIG. 1 is a graph showing the effect of extraction temperature on protein yields. All extractions were done with 3% ammonium hydroxide at pH=10.5 after an AFEX treatment. The results were combined after two (2) separate extractions using 11:1 liquid/solid ratio and 3 minute residence time. All runs were done in duplicate and error bars represent the maximum and minimum values.

FIG. 2 is a graph showing the effect of ammonia concentration on protein yields. All extractions were done at 50° C. and at pH=10.5 after an AFEX treatment. The results are combined after two (2) separate extractions using 11:1 liquid/solid ratio and 3 minute residence time. All runs were done in duplicate and error bars represent the maximum and minimum values.

FIG. 3 is a graph showing the effect of extraction pH on protein yields. All extractions were done with 3% ammonium hydroxide and at 25° C. The results are combined after two (2) separate extractions using 11:1 liquid/solid ratio and 3 minute residence time. All runs were done in duplicate and error bars represent the maximum and minimum values.

FIG. 4 is a graph showing effect of reducing agents on protein yields for untreated and AFEX treated samples. All extractions were done with 3% ammonia by weight, 25° C., and at pH=10.5. The results are combined after two (2) separate extractions using 11:1 liquid/solid ratio and 30 minute residence time. Both the ionic sodium dodecyl sulfate (SDS) and the nonionic Tween 80 (Tw80) surfactants were tested, both with and without the addition of β-mercaptoethanol. All runs were done in duplicate and error bars represent the maximum and minimum values.

FIG. 5 is a graph showing amino acid profiles for untreated protein extract, AFEX treated protein extract, and the native switchgrass protein.

FIG. 6A is a process flow diagram for AFEX treatment with extraction prior to hydrolysis. Balances around the protein and ash content are given, as well as total mass and the amount of glucose and xylose produced.

FIG. 6B is a process flow diagram for AFEX treatment with extraction after hydrolysis. Balances around the protein and ash content are given, as well as total mass and the amount of glucose and xylose produced.

### DESCRIPTION OF THE PREFERRED EMBODIMENTS

All patents, patent applications, government publications, government regulations, and literature references cited in this specification are hereby incorporated herein by reference in their entirety. In case of conflict, the present description, including definitions, will control.

The term “AFEX” means Ammonia Fiber Expansion or Explosion. The fibers are opened in the process to expose the proteins and structural carbohydrates.

By disrupting the lignocellulosic structure of the biomass, proteins appear to more easily diffuse out of the biomass and into the solution. It may be possible to further increase yields of sugar and protein by further integration of pretreatment, extraction, and hydrolysis. Removing soluble material during extraction can remove hydrolysis inhibitors, whereas hydrolysis of the cellulose and hemicellulose can further improve protein recovery. One (1) particular advantage of integration is in the use of a dilute ammonia solution as an extraction agent. A portion of the ammonia used in the AFEX process can be diluted and used as the extraction solution before returning to the ammonia recovery system, potentially lowering overall raw material requirements.

In particular, the feasibility of extracting proteins from switchgrass harvested in the spring while simultaneously producing sugars through enzymatic hydrolysis was examined. The optimal conditions for solid/liquid extraction using aqueous ammonia were determined and compared to other solvents. Potential process flow schemes were examined with respect to their sugar and protein yields before a complete material balance of the final process was determined.

### Materials and Methods

### Feedstock

The feedstock used in this experiment was Alamo switchgrass obtained from Auburn University and harvested on May 22, 2005. The moisture content of the material was approximately 9%. All material was ground to less than 2 mm prior to experiments.

### Pretreatment

The AFEX pretreatment was performed in a 300 mL stainless steel pressure vessel. Water was mixed with the switchgrass to increase the moisture content to 80% dry weight basis. Glass spheres were added to minimize void space, thereby reducing the amount of ammonia in the gaseous state. The lid was bolted shut, and a sample cylinder loaded with 1 (+/−0.04) g NH₃ per g dry biomass, allowing the ammonia to be charged into the vessel. The reactor was heated using a 400 W PARR heating mantle, and allowed to stand at 100° C. (+/−1° C.) for five minutes. The pressure was explosively released by rapidly turning the exhaust valve. The treated samples were removed and were placed in a fume hood overnight to remove residual ammonia.

### Hydrolysis

The enzymatic hydrolysis procedure was based upon the LAP-009 protocol from the National Renewable Energy Laboratory (19). Samples were hydrolyzed in Erlenmeyer flasks at 10% solid loading buffered to pH 4.8 by 1 M citrate buffer. Spezyme CP (Genencor; Palo Alto, Calif.) cellulase was loaded at 15 FPU/g glucan (31 mg protein/g glucan), and β-glucosidase (Novozyme 188; Bagsvaerd, Denmark) at 64 pNPGU/g glucan. All samples were incubated at 50° C. with 200 rpm rotation. Sugar concentration after 168 hours was determined using a Waters High Performance Liquid Chromatograph (HPLC) system equipped with a Bio-Rad (Richmond, Calif.) Aminex HPX-87P carbohydrate analysis column. Degassed HPLC water with a flow rate of 0.6 mL/min was used as the mobile phase, while the temperature in the column was kept constant at 85° C.

### Protein Extractions

Screening for optimal protein extraction conditions was done using a Dionex (Sunnyvale, Calif.) ASE 200 Accelerated Solvent Extractor. Extractions were performed at 1500 psi, which reduces the required residence time from 30 to 3 minutes. Extractions were done using 11:1 (w/w) liquid/solid ratio and two (2) separate extractions per sample. For experiments involving varying the pH, hydrochloric acid was used to reduce the pH. The pH of the solution was measured after the extraction was complete. Once the optimal extraction conditions were obtained, all further extractions were performed in flasks for 30 minutes with a 10:1 liquid/solid ratio while continuously stirred.

Due to the presence of ammonia nitrogen, both during the AFEX pretreatment and subsequent extractions, it is impossible to use standard nitrogen analysis methods (the Kjehldahl or Dumas methods) to measure total protein content. Instead, protein concentration was measured using a Pierce (Rockford, Ill.) bichronimic acid colorimetric assay kit using bovine serum albumin (BSA) as a standard. To reduce the effects of interfering agents such as ammonium salts, lignin components, and glucose, the proteins were first precipitated and resolubilized (20). A 100 μL 0.15% sodium deoxycholate was added to 100 μL protein solution and allowed to sit for 15 minutes. 200 μL of 15% trichloroacetic acid solution was added, and allowed to sit at 2° C. overnight. The mixture was centrifuged at 13000 RPM for 10 minutes, and the resulting pellet washed with acetone. The pellet was resolubilized in a buffer solution containing 0.1M Tris, 2.5M urea, and 4% SDS. Known concentrations of protein extracts were used to calibrate the protein recovery of this method.

### Composition Analysis

The weight and moisture content of the remaining solid fraction after each processing step was measured for determining the mass balance in the system. The composition of each of these fractions was determined based upon NREL's LAP 002 protocol (19). Ash content was determined by heating 1.5 g of biomass at 575° C. for 24 hours and measuring the weight loss. Water and ethanol extractives were removed using a soxhlet extraction. A portion of the extracted biomass was digested in concentrated (72%) sulfuric acid in a 10:1 liquid:solid ratio at 30° C. for one hour. The solution was diluted to 4% sulfuric and autoclaved at 120° C. for one hour, and then analyzed for sugar components using a Bio-Rad (Richmond, Calif.) Aminex HPX-87H HPLC column using sulfuric acid as the mobile phase. The acid insoluble lignin was measured as the remaining solid after hydrolysis less the ash content in the solid residue.

### Results and Discussion

### Composition Analysis

The composition of the switchgrass used in this study is shown in Table 1. Approximately 80% of the mass is accounted for. The remaining material is primary water soluble components, such as minor organic acids, and acid soluble lignin. The amount of protein present was lower than reported in literature for other strains of switchgrass (21). Switchgrass grown as a biomass energy crop and harvested early in the growing season would likely have protein contents near 10%, and thus, might be more suitable for integrated protein and sugar processing. The amount of fiber present is lower than switchgrass harvested at a later date, which seems to suggest lower sugar yields would also result from using an earlier cut. However, early cut switchgrass is less recalcitrant than that harvested in the fall, and thus, the lower cellulose and hemicellulose content may not be a significant factor. The low amount of lignin is a promising sign, as this implies less interference with hydrolysis as well as fewer harmful degradation products that could inhibit sugar production or otherwise be present in the protein product. Ash content is higher than at later harvests, as expected. It will likely be necessary to return much of this ash to the land in order to maintain a high quality soil.

| Component | % Value |
|---|---|
| Glucan | 26.4 |
| Xylan | 16.4 |
| Arabinan | 3.5 |
| Sucrose | 3.4 |
| Protein | 7.3 |
| AI Lignin | 10.8 |
| Lipids | 7.3 |
| Ash | 4.8 |
| Total | 79.9 |

Table 1 shows the composition of Alamo (g/100 g dry matter) switchgrass. AI-acid insoluble

The essential amino acid profile for switchgrass, along with literature values for corn and soy (22), is shown in Table 2. The most promising feature of switchgrass protein is the high value seen for lysine, an essential amino acid that is often the first limiting amino acid in poultry diets. High values for phenylalanine and valine are also seen. Although switchgrass is somewhat deficient in leucine, arginine, and methionine, these amino acids are relatively abundant in corn. Thus, a corn-switchgrass protein diet would balance out these deficiencies, and thus might be a strong alternative to a corn-soy diet.

Table 2 shows essential amino acid profile of Alamo switchgrass (SG) compared to literature values for soybean and corn grain (22). Values are in g amino acid100 g protein. Of particular note are lysine, phenylalanine, and valine, of which switchgrass is rich in, and methionine, of which switchgrass is somewhat deficient.

| | Arg | His | Ile | Leu | Lys | Met | Phe | Thr | Val |
|---|---|---|---|---|---|---|---|---|---|
| SG | 2.1 | 1.8 | 3.7 | 5.6 | 7.4 | 0.6 | 9.1 | 4.9 | 6.1 |
| Soy | 7.5 | 2.6 | 4.9 | 7.7 | 6.1 | 1.6 | 5.1 | 4.3 | 5.1 |
| Corn | 2.9 | 1.6 | 4.3 | 16.2 | 1.6 | 2.3 | 5.9 | 3.1 | 4.4 |

### Extraction Optimization

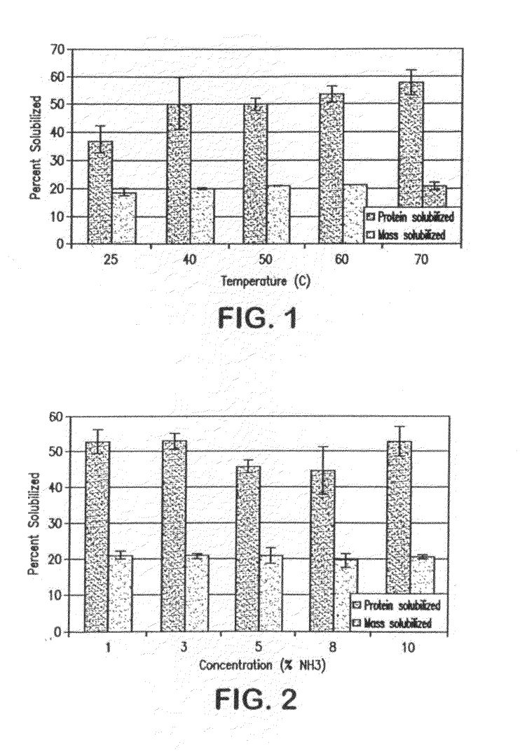

FIG. 1 shows the effect of the temperature of the extraction on the overall protein and mass yields. Protein yields increased significantly from 25° C. to 40° C., but further increases in temperature did not result in major improvements in protein yield. It is likely that most, if not all, of the proteins present in the switchgrass are in their natural state, as the harvesting and drying conditions should not have damaged them. As such, the mild temperatures should not unfold the proteins or significantly affect their solubility.

The effect of ammonia concentration on extraction yields is seen in FIG. 2. Protein yield remains constant from 1-3% NH4+, but then begins to drop off. This is most likely due to “salting out” the protein, as the increase in salt concentration decreases the amount of water available to solubilize the protein. There does not appear to be any salting in effect, likely because 1% salt solution is already a sufficient concentration to solubilize the protein. The total mass solubilized was unaffected by salt concentration, as expected.

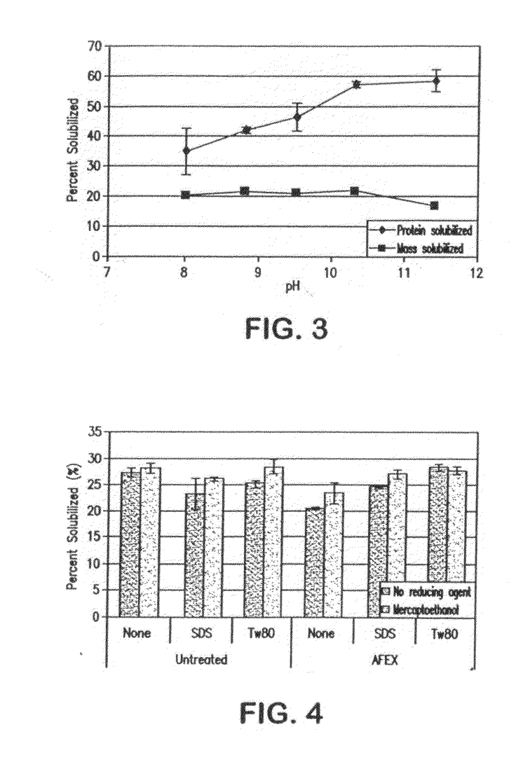

The most significant factor in determining protein yields is the pH of the system, as seen in FIG. 3. The amount of protein extracted increased dramatically from a pH of 8 to 10.5 before leveling off. Similar trends have been seen in other types of biomass (10-16). Most proteins have an acidic isoelectric point, the pH at which the protein will have no net charge and therefore, be the least soluble in a polar medium. Thus, increasing the pH should increase protein solubility, as demonstrated here. The most alkaline solution also produced a significant drop in the total mass solubilized, a potentially useful characteristic. If there is less biomass in solution, it should be easier to purify the proteins. In addition, the biomass lost during extraction likely includes hemicellulose that could be hydrolyzed into sugars for ethanol production. Further increases in pH would require a stronger base than ammonia and might degrade the protein, and thus were not considered.

As seen in FIG. 4, attempts were made to improve yields by the addition of the nonionic surfactant Tween 80, the ionic surfactant sodium dodecyl sulfate (SDS), and β-mercaptoethanol, a reducing agent. No significant improvements can be found by the addition of either surfactant or reducing agent for the untreated switchgrass. However, adding β-mercaptoethanol and Tween 80 to AFEX treated grass did increase protein removal. This would seem to suggest that the AFEX process affects the proteins in some manner. This effect might be through the creation of sulfur-sulfur bonds, which would then be cleaved by β-mercaptoethanol, or by proteins unfolding and exposing hydrophobic sites, which can be resolubilized with surfactants. The total mass solubilized also increased with the addition of surfactants, such as Tween 80, most likely due to interactions between the surfactants and hydrophobic portions of the biomass.

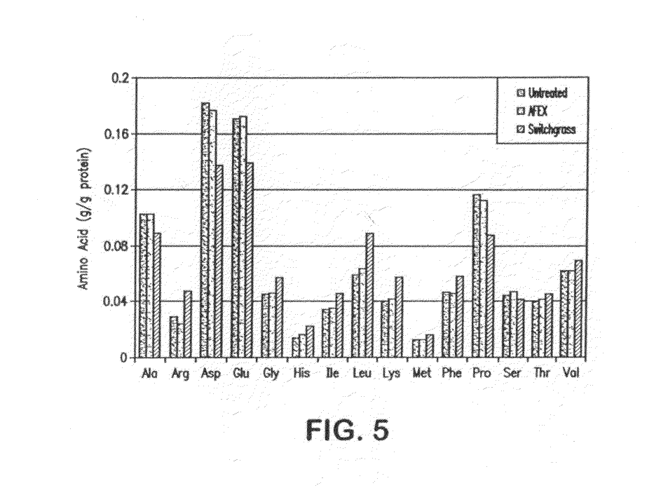

To determine whether AFEX pretreatment affects the types of proteins recovered, the composition of the individual amino acids was determined, as seen in FIG. 5. Both the untreated and AFEX treated samples were extracted at the optimal ammonia conditions without adding surfactant or reducing agent. Although the amino acid profile for the proteins solubilized during extraction compared to the total protein from switchgrass is quite different, there is very little difference between extractions from untreated and AFEX treated grass. Although AFEX does disrupt the cellular structure of the biomass, it does not appear to release any other proteins to be available for extraction. Therefore, it appears that the structure of the plant is not a major hindrance in protein recovery, but rather the structure of the protein itself.

Thus, optimal extraction conditions for switchgrass are approximately 3% aqueous ammonia at a pH of 10 and temperature of 40-50° C. These conditions are in line with those seen for protein extraction of other types of biomass, and are the conditions used for all subsequent experiments reported here. Total protein yields are approximately 40%. However, AFEX did not appear to significantly improve yields of protein, unlike previously reported with coastal bermudagrass and sodium hydroxide (17).

### Integration

Two (2) potential scenarios for integrated sugar and protein recovery were studied: an extraction immediately after AFEX and an extraction immediately after hydrolysis. A third option, extraction prior to AFEX, produced sugar yields far below the first two scenarios, and so is not presented here. It is possible that extracting proteins and other material prior to AFEX changes the effects of AFEX pretreatment. AFEX produces some organic acids that may inhibit hydrolysis, and it is possible that a prior extraction could produce more of these inhibitory acids. Washing the biomass after AFEX increased the sugar yields to approximately the same level as hydrolysis without any previous extraction. However, this process was deemed to require too much water use with no clear advantage, and thus was not studied in greater depth.

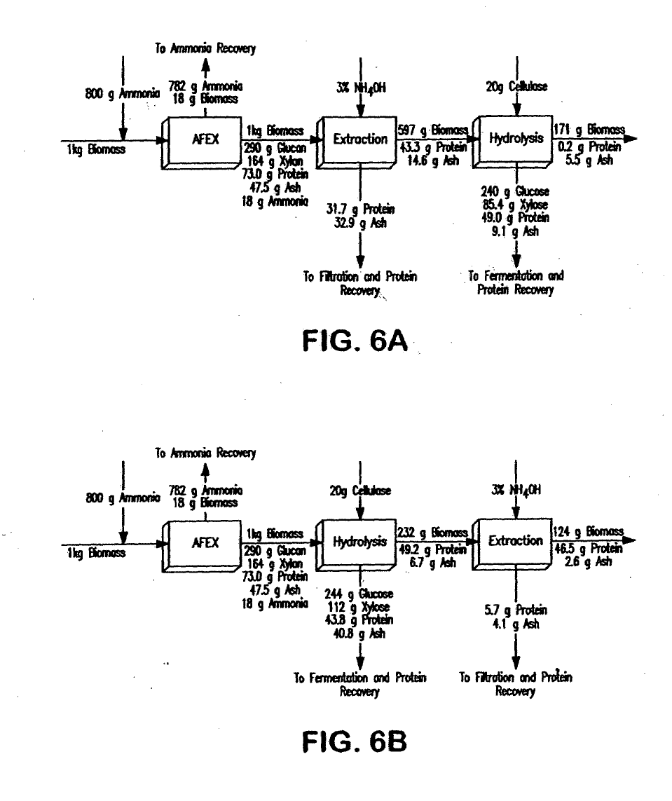

The overall mass balance for integrated sugar and protein with extraction prior to hydrolysis is seen in FIG. 6A. Final yields were 240 g glucose, 85.4 g xylose, and 80.7 g protein per kg dry biomass. Sugar recovery was approximately 74% of theoretical values, indicating further improvements in sugar recovery can be made. Approximately 40% of the protein was found in the extract and 60% in the hydrolysate, demonstrating that protein must be recovered from both streams in order to be economical. It should be noted that the insoluble biomass was washed after hydrolysis to insure all soluble components were recovered, and thus this may have acted as a second extraction to remove any remaining proteins bound to insoluble portions of the biomass. Total protein yield is approximately 87% of the total, taking into account both the switchgrass protein and the enzymes used in hydrolysis. However, virtually no insoluble protein remains in the biomass, thus suggesting that the remaining protein was broken down and lost at some point during the process.

Approximately 40% of the biomass is solubilized during the initial protein extraction step. It may be possible to utilize this soluble fraction of the biomass after the proteins have been removed. The protein might be concentrated and removed through ultrafiltration or heat precipitation, while the remaining solution undergoes further processing.

Most of the ash was removed from the biomass during the first extraction step. It is important to remove this ash, as the final insoluble residue would likely be burned to provide heat and power for the refinery. The ash content in switchgrass, particularly potassium, has been shown to cause problems with slagging in coal/biomass co-firing power plants. The remaining biomass contains only 3% ash, and thus should reduce this risk in heat and power generation. It remains to be seen if the ash in the extraction step can be separated and returned to the land. The fact that most of the ash is removed during one unit operation should help keep the costs of any ash processing step low, as only one stream needs to be treated.

Approximately 17% of the biomass remains insoluble throughout this process. There is virtually no protein or ash still present in this residue, which is mostly composed of unhydrolyzed fiber and insoluble lignin. This material would likely be burned for heat and power generation in the refinery, thus reducing natural gas or coal requirements. The lack of protein and ash would reduce the presence of NOx formation and slagging, respectively.

A separate balance, focusing on performing hydrolysis prior to extraction, is shown in FIG. 6B. Here, sugar yields were slightly higher, with a total of 356 g compared to 325 g per kg biomass using the previous approach. This is mainly due to xylan conversion, indicating that xylan oligomers were likely extracted along with protein during the initial extraction step in the previous scenario. However, although approximately 60% of the protein in the switchgrass was solubilized during hydrolysis, very little was extracted afterwards. During hydrolysis, other compounds may be produced that interfere with the calorimetric analysis, thus increasing the error involved. This mass balance, however, relies solely on the individual amino acids rather than a colorimetric response, and thus is a more accurate representation of actual protein levels. Subsequent extractions on the final residue did not release more than a small fraction of the residual proteins, making it unlikely that further treatments can remove the residual protein.

The amount of insoluble material remaining is less than that of the previous scenario, indicating that less heat and power can be produced. Although less ash is present, there is still a great deal of protein remaining. Protein has lower energy content than lignin and also its combustion will generate NOx. Thus, due primarily to the higher protein yields, an extraction prior to hydrolysis is preferred despite the slightly lower sugar yields.

### CONCLUSION

The experimental results show that the integrated recovery of sugar and protein from early cut switchgrass appears to be a feasible approach to a cellulosic biorefinery. Ammonium hydroxide has been shown to be an effective solvent for removing proteins from the biomass, thus opening up possibilities of integrating with AFEX pretreatment or providing a nitrogen source during fermentation. Integrating sugar and protein production will cause some tradeoffs, as producing maximum sugar will result in a lower protein recovery and vice versa. However, there are possibilities for overcoming these obstacles.

Further integration of these two (2) steps is also possible. If the loss in sugar yields is due solely to oligomer loss, then using the protein extract as the hydrolysate liquid after separating the proteins would reduce these losses. This would require neutralizing the extract, but would decrease overall water use and thereby improve the environmental and economic performance of the refinery. In addition, the fact that there are multiple protein streams may allow further specialization. If the cellulase enzymes are still active after hydrolysis, it may be possible to concentrate and recycle them, again reducing operating costs.

It still remains to be seen if downstream processing can fully separate the proteins and sugars in order to produce the desired final products. This requires separating the proteins from the remaining sugar and other soluble portions of the biomass, likely through ultrafiltration.

This invention is aimed at collecting the protein content found within grasses and optionally as those proteins added during cellulose and hemicellulose hydrolysis using dilute ammonium hydroxide as the solvent. These proteins are captured in two steps: the initial hydrolysis of the carbohydrates and a separate extraction step where the order is dictated by economics. Thus, proteins are recovered from the hydrolysate before or after the carbohydrates are fermented. The remaining biomass after fermentation then undergoes a simulated crossflow extraction to remove any remaining proteins.

This is the first method to use ammonium hydroxide as a solvent, which has two (2) advantages over the previous approaches. First, any residual ammonia remaining on the final protein product used as a feed for ruminants would count as extra nitrogen in its diet, thereby improving the overall crude protein content of the final product. Other alkaline solutions could provide a negative effect due to the presence of unwanted ions such as sodium. Second, ammonia is also used during the Ammonia Fiber Explosion (AFEX) pretreatment process. Thus, the ammonia used for extraction can be taken from the ammonia recovery system in place for the AFEX process, and then recycled back into AFEX after concentrating the proteins. Thus, using ammonia for extraction eliminates the need for an additional reagent.

The process can remove over 99% of the proteins from the solid biomass, indicating a very high recovery is possible. Extracting proteins from untreated switchgrass provides yields of approximately 35%. By using a separate extraction step after hydrolysis, it is possible to recover not only the proteins still remaining within the biomass, but also those that are adsorbed onto the biomass surface. In addition, the disruption of the biomass' structure during the AFEX pretreatment process and the carbohydrate hydrolysis improves the diffusion of proteins from the solid into solution. No other process has focused on combining protein extraction with AFEX and carbohydrate hydrolysis.

With two (2) separate protein streams, there exists the possibility that they can be used for separate purposes. For example, the stream containing the enzymes required for hydrolysis can be recycled, thus reducing the overall cost of carbohydrate production. The other proteins within that stream would bind to the lignin present, deactivating those sites and preventing the enzymes from binding to them. This could potentially increase the rate of hydrolysis, further reducing the cost to the refinery.

A simulated crossflow extraction is used to increase the overall amount of proteins extracted while still keeping solvent use low. The biomass is put through a number of extractions while still maintaining a small solvent use by using the same solvent for subsequent extractions, as only the final extraction uses fresh solvent. This not only reduces the cost of extraction, but also the costs to concentrate the proteins downstream.

This process is useful for a cellulosic ethanol production facility, as it could provide a valuable co-product to ethanol. These proteins can be sold as animal feed, serving as a substitute for soy protein. In addition, it is possible, to apply this method to transgenic biomass engineered to produce specific industrial or pharmaceutical enzymes, as described in U.S. application Ser. No. 11/489,234, filed Jul. 19, 2006, and U.S. Pat. No. 7,049,485 which are commonly owned by the Assignee and which are incorporated herein by reference in their entireties.

This method can be implemented in line with cellulose hydrolysis. No changes would be necessary for either the AFEX process or the hydrolysis reaction chamber. The solids and liquids must be separated after hydrolysis, either through centrifugation or standard filtration. The liquid stream then can pass through a crossflow ultrafiltration system, allowing the sugars and most of the water to pass through, leaving behind a concentrated protein product.

A simulated crossflow extraction would need to be implemented for the remaining solid material. The solids would pass through three (3) separate extraction vessels, where they would be mixed with the incoming ammonium hydroxide. The solids and liquids will need to be separated between each step, again through either centrifugation or filtration. After the solvent undergoes its final extraction step, it must also be concentrated. It can be combined with the liquid stream from the hydrolysate or be concentrated through a separate crossflow ultrafiltration step.

The remaining ammonium hydroxide solution can then be recycled into the AFEX ammonia recovery system. It may be necessary to remove any organic matter still remaining in solution before this step. A simple distillation column can remove the volatile ammonia, concentrating and separating it from the solubilized biomass. This stream can then be recovered, while the remaining liquid can be sent elsewhere for waste treatment or further processing.

A few other alternatives are available, depending on how integrated one wishes this process to be. Rather than recycling the ammonia into the AFEX recovery process, a separate recycle stream for the extraction process can be used. If the extraction is performed prior to hydrolysis, the ammonium hydroxide solution can also be neutralized and used as the hydrolysate media as well. A standard one or two step extraction process can replace the simulated crossflow extraction.

Cellulosic biomass contains large amounts of structural carbohydrates (cellulose, hemicellulose, etc.) that might provide much less expensive sugars for fermentation or non-biological transformation to a variety of products or as improved animal feeds. Such biomass also contains smaller but nonetheless significant amounts of proteins and other solubles such as simple sugars, lipids and minerals. These less abundant components can be separated from the structural carbohydrates as part of a larger “biorefining” process. Recovering these soluble components during biorefining reduces the amount of waste that must be handled by the biorefinery and would also help provide additional valuable products that could improve the economic feasibility of the overall biorefining process. In addition, plants may be genetically engineered to produce various molecules that might be separated and recovered from herbaceous biomass in this way.

The specific features of this invention that make it more advantageous than old methods are as follows: (1) it strives to extract and utilize all solubles in herbaceous biomass, not just protein; (2) it can utilize all types of herbaceous biomass, including both wet and dry biomass, not just freshly harvested materials; (3) it integrates easily and naturally into a larger process using concentrated ammonia to treat biomass to enhance the conversion of cellulose and hemicellulose to sugars; (4) the conditions of solubles recovery (pH and temperature) can preserve much of the value of fragile molecules, including proteins; and (5) separating and upgrading these solubles to make salable products avoids the expense and other difficulties associated with treating them as wastes and may significantly improve the economic “bottom line” of the overall process.

Markets that might use this invention include: (1) the U.S. chemical industry which is beginning to move away from petroleum as a source of chemical feedstocks and is interested in inexpensive sugars as platform chemicals for new, sustainable processes; (2) the fermentation industry, especially the fuel ethanol production industry which is also interested in inexpensive sugars from plant biomass; (3) the animal feed industry which is strongly affected by the cost of protein and other nutrients for making animal feeds of various kinds; and (4) the fertilizer industry that may utilize the minerals that will result from solubles extraction.

The steps are generally:

(1) Following pretreatment of herbaceous biomass with concentrated ammonia: water mixtures in an AFEX process to disrupt the chemical and physical structure of biomass.
(2) Soak the pretreated biomass in warm (up to 80° C.), alkaline (up to pH 10) aqueous solutions of ammonium hydroxide in water, using approximately 5-15 mass units of water per mass of dry biomass.
(3) Allow sufficient time for the desired level of extraction to occur under these conditions, but less than 1 hour.
(4) Using appropriate filtration equipment, remove the liquid from the solids.
(5) Acidify the liquid to about pH 5.0 or thereabouts and/or heat the liquid stream to precipitate proteins and other less soluble components.
(6) Recover and separate these proteins and associated solubles by appropriate combinations of washing, drying and ultrafiltration.
(7) Treat the residual liquid remaining after protein precipitation or separation to prepare it to serve as a microbial growth stimulant.
(8) Enzymatically hydrolyze the residual solids from which these proteins were extracted to release simple sugars for fermentation and treat the resulting liquid to recover additional protein and other non-sugar solubles if the concentrations of these species warrant it.

Efficient, mature biomass refining to fuels and chemicals requires complete utilization of all components of the biomass, including protein and other solubles. These additional products help improve the overall economics of biomass refining and avoid the costs associated with treating these components as wastes if they are not recovered in useful products.

Lignocellulosic biomass, especially herbaceous biomass, contains significant amounts of protein and other solubles. This invention addresses the opportunity to integrate recovery of solubles such as protein in an overall biomass refining system. Warm solutions of ammonia and water are used to extract this protein and other solubles from biomass. The extracted species are recovered and sold as additional products from the biorefinery, thereby increasing profits and reducing the amount of waste that would otherwise be treated.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### REFERENCES

1. Lin, Y. and Tanaka, S. (2006), Appl. Microbiol. Biotechnol. 69, 627-642.
2. Greene, N. (2003), Growing Energy, Natural Resources Defense Council.
3. Sanderson, M. A., Reed, R. L., McLaughlin, S. B., Wullschleger, S. D., Conger, B. V., and Parrish, D. J. (1996) Bioresour. Technol. 56, 83-93.
4. Mosier, N., Wyman, C., Dale, B Elander, R., Lee, Y., Holtzapple, M., and Ladisch, M. (2005), Bioresour. Technol. 96, 673-686.
5. Sulbaran-de-Ferrer, B., Aristiguieta, M., Dale, B., Ferrer, A., and Ojeda-de-Rodriguez, G. (2003), Appl. Biochem and Biotechnol. 105-108, 155-164.
6. Teymouri, F., Laureano-Perez, L., Alizadeh, H., and Dale, B. (2005), Bioresource Technology 96, 2014-2018.
7. Holtzapple, M., Jun, J., Ashok, G., Patibandla, S., Dale, B. (1991), Appl. Biochem and Biotechnol. 28-29, 59-74.
8. Ferrer, A., Byers, F. M., Sulbarán De Ferrer, B., Dale, B. E., and Aiello, C. (2000), Appl. Biochem. and Biotechnol. 84-86, 163-179.
9. Alizadeh, H., Teymouri, F., Gilbert, T., and Dale, B. (2005), Appl. Biochem. and Biotechnol. 121-124, 1133-1141.
10. Ordonez, C., Asenjo, M., Benitez, C., and Gonzalez, J. (2001), Bioresour. Technol. 78, 187-190.
11. Lawhon, J. (1986), U.S. Pat. No. 4,624,805. Nov. 25, 1986.
12. El-Adaway, T., Rahma, E., El-Bedawey, A., and Gafar, A. (2001), Food Chem. 74, 455-462.
13. Park, S. and Bean, S. (2003), J. Agric. Food Chem. 51, 7050-7054.
14. Betschart, A. and Kinsella J. (1973), J. Agric Food Chem., 21(1), 60-65.
15. Fernandez, S., Padilla, A., Mucciarelli, S. (1999), Plant Foods for Human Nutrition 54, 251-259.
16. Fiorentini, R. and Galoppini, C. (1981), J. Food Sci. 46, 1514-1520.
17. De La Rosa, L. B., Reshamwala, S., Latimer, V., Shawky, B., Dale, B., and Stuart, E. (1994), Appl. Biochem. and Biotechnol. 45-46, 483-497.
18. Urribarrí, L., Ferrer, A., and Colina, A. (2005), Appl. Biochem. and Biotechnol. 121-124, 721-730.
19. NREL (2004), Chemical Analysis and Testing (CAT) Standard Procedures, National Renewable Energy Laboratory.
20. Lovrien, R. and Matulis, D. (1995), Current Protocols in Protein Science, 3.4.1-3.4.24.
21. Madakadze, I., Stewart, K., Peterson, P., Coulman, B., and Smith, D. (1999) Crop Sci. 39, 552-557.
22. Allan, G., Parkinson, S., Booth, M., Stone, D., Rowland, S., Frances, J., and Warner-Smith, R. (1999)., Aquaculture 186, 293-310.

We claim:

1. A process for extracting plant proteins from a lignocellulosic plant biomass comprising:
(a) providing a harvested lignocellulosic plant biomass;
(b) treating the plant biomass with an Ammonia Fiber Explosion (AFEX) process to provide a treated plant biomass; and
(c) extracting proteins in the treated plant biomass with an aqueous alkaline ammonium hydroxide solution comprising up to about 3% by weight NH₄OH to provide the extracted proteins in the solution.

2. The process of claim 1 wherein the plant is a monocot.

3. The process of claim 2 wherein the monocot is rice or maize.

4. The process of claim 1 wherein the plant material is switchgrass.

5. The process of any one of claims 1, 2, 3 or 4 wherein the pH is above about 8.

6. The process of any one of claims 1, 2, 3 or 4 wherein the proteins are separated from the solution by precipitation or ultrafiltration.

7. The process of any one of claims 1, 2, 3 or 4 wherein the extracting is after a hydrolysis step in the plant biomass, after step (b), to produce sugars from sugar precursors in the biomass.

8. The process of any one of claims 1, 2, 3 or 4 wherein the extracting of the proteins is before a hydrolysis step in the plant biomass, after step (b), to produce sugars from sugar precursors in the biomass and optionally in addition after the hydrolysis step.

9. A process for isolating plant proteins from a lignocellulosic plant biomass comprising:
(a) providing a harvested lignocellulosic plant biomass;
(b) treating the plant biomass with an Ammonia Fiber Explosion (AFEX) process to provide a treated plant biomass;
(c) soaking the treated plant biomass in an alkaline aqueous solution of ammonium hydroxide at 25° to 70° C. to provide a soaked plant biomass in the solution;
(d) extracting the solution from the soaked plant biomass in step (c); and
(e) separating crude proteins from the solution of step (d) so as to provide isolated plant proteins from the plant biomass.

10. The method of claim 9 wherein the plant is a monocot.

11. The method of claim 10 wherein the monocot is switchgrass, rice or maize.

12. The process of claim 9 wherein the plant biomass is switchgrass.

13. The process of any one of claims 10, 11, 12 or 13 wherein the pH is above about 8.

14. The process of any one of claims 9, 10, 11 or 12 wherein the proteins are separated from the solution by precipitation or ultrafiltration.

15. The process of any one of claims 9, 10, 11 or 12 wherein the proteins are separated after a hydrolysis step in the plant biomass, after step (b), to produce sugars from structural carbohydrates in the biomass.

16. The process of any one of claims 9, 10, 11, 12 or 13 wherein the proteins are separated before a hydrolysis step in the biomass, after step (b), to produce sugars from structural carbohydrates and optionally in addition after the hydrolysis step.
